(19) United States
(12) Patent Application Publication
(10) Pub. No.: US 2010/0076305 A1
(43) Pub. Date: Mar. 25, 2010

(54) METHOD, SYSTEM AND COMPUTER PROGRAM PRODUCT FOR TARGETING OF A TARGET WITH AN ELONGATE INSTRUMENT

(75) Inventors: Lena Maier-Hein, Heidelberg (DE); Alexander Seitel, Waldshut (DE); Ivo Wolf, Wiesenbach (DE); Hans Peter Meinzer, Heidelberg (DE)

Correspondence Address: DUANE MORRIS LLP - DC, 505 9th Street, Suite 1000, WASHINGTON, DC, 20004-2166, US

(73) Assignee: Deutsches Krebsforschungszentrum Stiftung des Offentlichen Rechts, Heidelberg (DE)

(21) Appl. No.: 12/481,774

(22) Filed: Jun. 10, 2009

Related U.S. Application Data
(60) Provisional application No. 61/075,467, filed on Jun. 25, 2008.

(51) Int. Cl. A61B 5/05

(52) U.S. Cl. 600/426

(57) ABSTRACT

An embodiment is directed to a method and a system for assisting the targeting of a target with an elongate instrument, wherein the instrument is to be inserted into a living object's body part along a predetermined trajectory extending between an entry point of said instrument into said body part and a target point associated with said target. The method comprises an instrument directing assisting step for generating and displaying an image allowing a user to assess to which extend the longitudinal axis of the instrument is aligned with the vector connecting the target point and the tip portion of said instrument. Also, the method comprises an instrument guiding assisting step of generating and displaying an image allowing a user to assess to which extent the instrument motion during insertion thereof coincides with the predetermined trajectory.

### FIELD OF THE INVENTION

The present invention relates to the field of medical technology, and in particular to methods systems for computer assisted targeting of a target in soft tissue.

### BACKGROUND OF THE INVENTION

Minimally invasive interventions such as biopsies or thermal ablation therapy of certain target areas such as tumours require that an elongate instrument is inserted into a living object's body part such as to exactly reach the target area. The elongate instrument can for example be a biopsy needle or a needle configured for thermal ablation, such as radiofrequency ablation. One of the main challenges related to the intervention is the placement of the instrument at exactly the envisaged position. This is especially true when the target is situated closely to risk structures such as large vessels, further tumours, organs, etc. which must not be hurt by the instrument upon insertion.

With reference to FIG. 1, a common prior art targeting method based on computed tomography (CT) is described. In the example of FIG. 1, it is assumed that the target is a tumour 10 in a human patient's liver 12. In a first step, a CT image shown in the first panel of FIG. 1 is taken to locate the tumour 10. While in the present example CT medical imaging is chosen, other medical imaging methods such as ultrasound or nuclear magnetic resonance (NMR) imaging could also be used.

After getting an idea about the approximate position and size of the tumour, a pre-interventional CT scan is made, for which markers 14 formed by a set of parallel needles are attached to the skin of the patient which are also visible in the CT image (step 2). By visually comparing the CT image and the patient, the markers 14 assist the physician in “mentally” registering the patient with the CT image. The CT image with a number of markers 14 is schematically depicted in the panel of step 3.

In a fourth step, the pre-interventional CT image is used for planning a desired trajectory 16, which extends between an insertion point 18 and a target point 20, which could for example be the centre of mass of the tumour 10. The predetermined trajectory 16 is chosen using the information of the pre-interventional CT image, in which risk structures which have to be avoided by the instrument can be seen. For reasons explained below, the predetermined trajectory 16 will typically be in a transverse body plane of the patient.

In the fifth step, the elongate instrument is inserted into the patient's body. To this end, the physician will typically place the tip of the instrument on the predetermined insertion point 18, which he or she will find more or less accurately by resorting to the markers 14, which are both visible in the CT image as well as on the patient's skin. The insertion point could for example be defined by a point lying in between two of the markers 14 shown in panel 2 of FIG. 1 and in the CT plane, which can be indicated by a laser beam. Again, the finding of the insertion point corresponds to a “mental” registering of the patient with the CT image. After the tip of the needle is placed where the physician believes the predetermined insertion point is, the needle is directed such as to point towards the tumour 10. If the planned trajectory 16 is located in a transverse plane, the physician will tilt the instrument within said plane such as to establish a given angle with regard to the sagittal plane, which angle can be determined from the transverse CT image.

After the physician has angled the instrument as deemed appropriate, he or she can insert the instrument typically in a number of consecutive partial insertion steps up to a predetermined depth corresponding to the length of the predetermined trajectory 16, which can also be discerned from the CT image.

In the sixth step, a further CT image is made, for controlling whether the correct trajectory has been found and whether the tumour 10 has been reached by the tip of the instrument shown as 22 in panel 6. If the targeting is found to have been successful, the biopsy or ablation can be performed. In the alternative, steps 5 and 6 and possibly step 4 have to be repeated.

As is apparent from the above description, this prior art targeting requires a lot of skill and experience, and in practice, even highly skilled physicians may need several attempts to finally reach the target properly, which makes the patient suffer from the repeated punctures with the elongate instrument as well as multiple exposures to radiation in the necessary control CT scans. Also, each correction increases the risk of tumour seeding and the probability that risk structures are accidentally damaged. Finally, while the targeting in the transverse plane already requires a considerable hand-eye-coordination by the physician, the intervention becomes even more difficult if the predetermined trajectory is in an arbitrary plane.

Thus, conventional methods of targeting remain very difficult and cumbersome.

### SUMMARY

This summary is provided to introduce a selection of concepts in a simplified form that are further described below in the detailed description. The summary is not intended to identify key features or essential features of the claimed subject matter, nor is it intended to be used to limit the scope of the claimed subject matter.

An embodiment of the invention is directed to a method for targeting of a target with an elongate instrument, in which the instrument is to be inserted into a living object's body part along a predetermined trajectory extending between an entry point into said body part and a target point associated with said target. According to an embodiment, the method comprises the following steps:
an instrument directing assisting step of generating and displaying an image allowing a user to assess to which extent the longitudinal axis of the instrument is aligned with a vector connecting the tip portion of said instrument and the target point, and
an instrument guiding assisting step of generating and displaying an image allowing a user to assess to which extent the instrument motion during insertion thereof coincides with said predetermined trajectory.

In one embodiment, the method further comprises an entry point finding assisting step of generating and displaying an image allowing a user to assess how the tip of the instrument has to be moved in order to approach the predetermined entry point.

According to this embodiment, the method comprises three steps, and in each step a suitable image for assisting the user in targeting is generated and displayed. The three steps of the method are specifically adapted to three crucial steps necessary in inserting the instrument to reach the target, namely the steps of placing the tip of the instrument at an entry point, directing the instrument such as to be aligned with a predetermined trajectory and inserting the instrument along the predetermined trajectory. In this embodiment, the entry point finding assisting step is a step for assisting the user to find an entry point that has been determined beforehand. This is an embodiment suitable for a case where the whole trajectory, such as a straight line connecting the entry point and the target, has been planned beforehand, and in which the aim is to insert the instrument as closely to the predetermined entry point as possible.

However, different embodiments are possible in which only the target point is predetermined and in which the entry point and the corresponding trajectory connecting the entry point and the target point is determined “on the fly”. For example, in one embodiment, the user could point the tip of an instrument to a trial insertion point and the trajectory connecting this trial insertion point and the target point could be computed, which then would amount to the “pre-determined trajectory” referred to in this disclosure. In one embodiment, information could be generated and displayed indicating whether the trial insertion point would be suitable, for example whether the corresponding trajectory would be sufficiently far away from risk structures or obstructing structures that have to be avoided with the tip of the instrument. Note that as far as the instrument directing assisting step and the instrument guiding assisting step are concerned, it makes no difference whether the trajectory has been planned before the intervention or is computed during the intervention, for example based on trial entry points.

In one embodiment, the image generated and displayed in the entry point funding assisting step represents a relative position between projections of said predetermined entry point and a tip portion of the instrument along a direction substantially parallel to the vector connecting the target point and the predetermined entry point onto a plane.

If only the projections of the tip portion and the predetermined entry point on a plane are displayed, the information displayed is only two-dimensional. However, this is exactly the two-dimensional information that is crucial upon finding the predetermined entry point. Namely, if the physician moves the needle tip closely above the skin of the patient looking for the entry point, the search is effectively two-dimensional, while the third component, i.e. a component parallel to the predetermined trajectory is obvious for the physician, since he knows that the entry point must be on the skin of the patient. By reducing the displayed information to the information that is actually needed in the step, the displayed image becomes very easy to understand and intuitive to interpret, as will be especially clear from an exemplary embodiment shown below.

The plane on which the tip portion and the entry point are projected could be a plane normal to said vector connecting said predetermined entry point and said target point; it could also be a plane in which the predetermined entry point is located. However, the invention is by no means limited to this choice. In particular, the projection can be a projection on any suitable plane, which does not even have to be flat. For example, a suitable plane for projecting on could also be the outer surface of the living object's body part. While in this embodiment, the displayed entry point finding assisting information is essentially two-dimensional, the distance from the surface of the skin, i.e. the third dimension may also be displayed.

Once the predetermined entry point is found, the instrument directing assisting step allows to easily tilt the elongate instrument such that its longitudinal axis is aligned with a vector connecting the target point and the tip portion of the instrument. It is advantageous to perform this directing or aligning step of the elongated instrument after finding the entry point, because the instrument can be pivoted around the contact point between its tip portion and the skin of the body part without loosing the entry point that has been found the previous step. Also, it is advantageous to perform this directing or aligning step prior to actually inserting the instrument into the body part, because an initial directional misalignment can not easily be corrected during insertion.

Again, in a preferred embodiment, the image generated and displayed in the instrument directing assisting step displays only two-dimensional information which is representative of the zenith angle and the azimuth angle of the longitudinal axis of the instrument with respect to a vector connecting the tip portion of the instrument and the target point. Perfect alignment is reached if the zenith angle becomes zero, while the azimuth angle information assists the user in recognizing which direction the elongate instrument has to be tilted to find alignment. In this regard, the definition of the image to be representative of a zenith angle and an azimuth angle shall impose no limitation other that there is a one-to-one correspondence between the two-dimensional image and the actual zenith and azimuth angles of the elongate instrument with regard to the optimal direction of insertion. A very intuitive graphical representation of such information is the projection of an end portion of the elongate instrument onto a plane perpendicular to a vector connecting the tip portion of the instrument and the target point along the direction defined by this vector, as will be shown in greater detail below.

In the third step, the instrument is to be inserted into the body part. The main task there is to maintain the proper alignment that had been found in the preceding step. In order to achieve this, in the instrument guiding assisting step an image is generated and displayed by which a user can assess whether the motion of the instrument during insertion coincides with the predetermined trajectory. An intuitive way to provide the user with this information is to generate and display an image corresponding to a view of a virtual camera placed at the tip of and directed along the longitudinal axis of the instrument. Simply speaking, in such a virtual view, the user has to steer the instrument during insertion such as to “fly” toward the target in said virtual image. The guiding during the insertion can be further assisted by displaying a tube- or tunnel-like structure coaxially surrounding the predetermined trajectory. This virtual tunnel makes it even easier for the user to “fly” toward the target along the predetermined trajectory. By the way, if in the first step, the user should have failed to exactly find the predetermined insertion point, the predetermined trajectory used in the instrument directing assisting step may be a corrected trajectory connecting the actual entry point and the target point.

While the virtual camera view allows to guide the instrument during insertion, it also helps to get a feeling for approaching the target, which assists in determining the proper time to stop the insertion. Stopping insertion can be further assisted by displaying a graphical representation of a parameter representing or related to the distance between the tip portion of the instrument and the target point.

In some embodiments, the virtual camera view used will be very sparse and only display the necessary information, such as the trajectory, the target and possibly the tube or tunnel surrounding it, such that the user will only have to concentrate on the relevant information for guiding the instrument upon insertion. However, in some embodiments, medical images of predetermined objects may be displayed. The medical images can be taken from an initial medical image used for planning of the trajectory, but they could also be provided by real-time imaging means. The predetermined objects can for example be objects that have to be avoided by the instrument, such as vessels, tumours, bony structures or organs such as lung, gall bladder etc. By displaying these predetermined objects, the user can be sure to avoid these structures during an insertion of the instrument.

In some embodiments, the method may comprise a step of tracking the instrument by optical and/or magnetic tracking means such as to continuously locate the position and orientation of the instrument in a tracking coordinate system. Further, the method may comprise a step of registering the tracking coordinate system with a coordinate system of a medical image of the body part. Such a registering step may comprise tracking of navigation aids, such as fiducials, which are provided on or are inserted to the body part and which are recognizable in the medical image or geometrically related with the medical image in some other way. In some embodiments, navigation aids comprising a needle-shaped body having an elongate portion serving as a marker portion can be used.

If the target is located in soft tissue which is not confined by rigid structures, such as bones, the target may move during intervention. In particular, if the target is located in the abdomen, the target may move due to the movement of the diaphragm during the breathing cycle of the patient. While the location of the target in the tracking system can in principle be determined by registering the image coordinate system with the tracking coordinate system, the calculated position of the target may deviate from the true position if the motion state of the body part deviates from the motion state in which the CT image used for registering had been taken.

According to one embodiment of the invention, such soft tissue motion can be accounted for during an initial registration step and optionally also during consecutive registering steps for real-time compensation of soft tissue motion. In one embodiment, the navigation aids may be tracked during a time interval during which the navigation aids may move along with the body part due to soft tissue motion. A motion state of the body part during this interval may be determined in which the positions of the navigation aids coincide best with their positions in the medical image. Then, an initial registration may be performed based on the tracked position of the navigation aids in said determined motion state. The rationale behind this embodiment is that a deviation of the motion state of the body part from the motion state in which the medical image was taken is reflected in a deviation of the tracked positions of the navigation aids from their positions in a medical image. Determining the motion state in which the positions of the navigation aids coincide best with their positions in the medical image thus allows to identify a motion state that is very close to the motion state of the body part upon taking the medical image. Then, the corresponding tracked positions of the navigation aids for that motion state can be used for the initial registration.

In some embodiments, a navigation aid comprising a needle-shaped body having an elongate portion serving as a marker portion may be used. Such a needle-shaped navigation aid or fiducial can be inserted fairly deeply into the body part to be close to the target and is thus well suited for reflecting the target motion.

In some embodiments, the method may comprise a step of repeated determining and displaying a value indicating how well the current positions of the navigation aids correspond with their positions in the medical image. An example of such a value, called fiducial registration error (FRE), will be described in more detail below. With the explanations given above, from this value the user can determine how well the current motion state of the body part coincides with the motion state of the medical image on which the registration of the target with the tracking coordinate system is based. In particular, the user can recognize certain motion states, such as certain periods of a breathing interval. Also, if the initial registration is performed in a motion state in which the FRE is small, during operation, the value may serve as a confidence value that the position of the target as calculated upon registering the medical image with the tracking coordinate system coincides with its current true position. Namely, if the current motion state of the body part leads to a small current FRE, this indicates that the motion state is similar to the one in which the initial registration has been performed, and accordingly, the position of the target calculated under the initial registration is expected to be valid for the current motion state. On the other hand, if the current FRE is large, this means that the initial registration, without further correction to account for soft tissue motion, may give a wrong position of the target for the current motion state. In this regard, it is emphasized that different definitions of FRE are applicable and that different types of transformations are possible, that lead to different values of the FRE. Any choice is possible, as long as the FRE associated with the transformation is able to reflect a deformation of the tissue. For example, the FRE could be an FRE associated with a rigid transformation.

Additionally or alternatively, the current position of the target point may be calculated based on information about the motion state of the body part. For example, a real-time deformation model can be applied which estimates the position of the target continuously from the positions of the tracked navigation aids.

The invention also relates to a system for targeting of a target. Various embodiments of such systems comprise means for carrying out some or all of the above method steps. Herein, the means can for example be a computer system, such as a personal computer, a notebook or a workstation, which is suitably programmed and which is connected to a display means, such as an ordinary computer display. Also, the invention relates to machine readable media having stored thereon instructions which when executed on a computer system allows to perform a method according to one of the embodiments described above.

### FIGURES

The accompanying drawings illustrate embodiments of the invention and, together with the description, serve to explain the principles of embodiments of the invention.

FIG. 1 is a schematic diagram illustrating the steps performed during targeting of the target with an elongate instrument according to prior art,

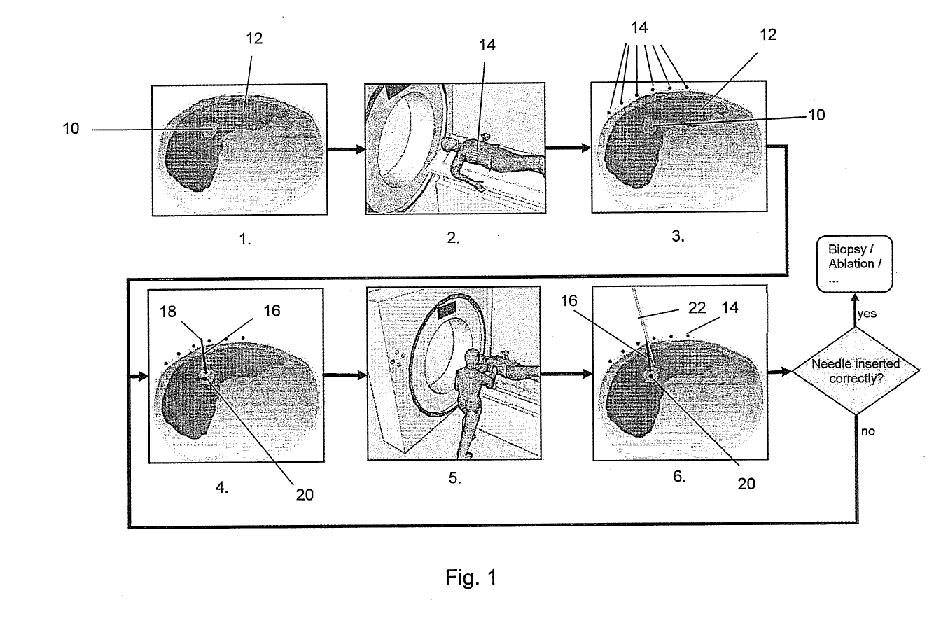

FIG. 2 is a schematic diagram illustrating the workflow of a method in which the invention may be employed,

FIG. 3 illustrates a screenshot corresponding to an assisted entry point finding step and a perspective diagram illustrating the geometry represented therein,

FIG. 4 illustrates a screenshot corresponding to an assisted instrument directing step and a perspective diagram illustrating the geometry represented therein, and

FIG. 5 illustrates a screenshot corresponding to an assisted instrument guiding step and a perspective diagram illustrating the geometry represented therein.

### DESCRIPTION OF THE PREFERRED EMBODIMENT

For the purposes of promoting an understanding of the principles of the invention, reference will now be made to the preferred embodiment illustrated in the drawings and specific language will be used to describe the same. It will nevertheless be understood that no limitation of the scope of the invention is thereby intended, such alterations and further modifications in the illustrated method and system and such further applications of the principles of the invention as illustrated therein being contemplated as would normally occur now or in the future to one skilled in the art to which the invention relates.

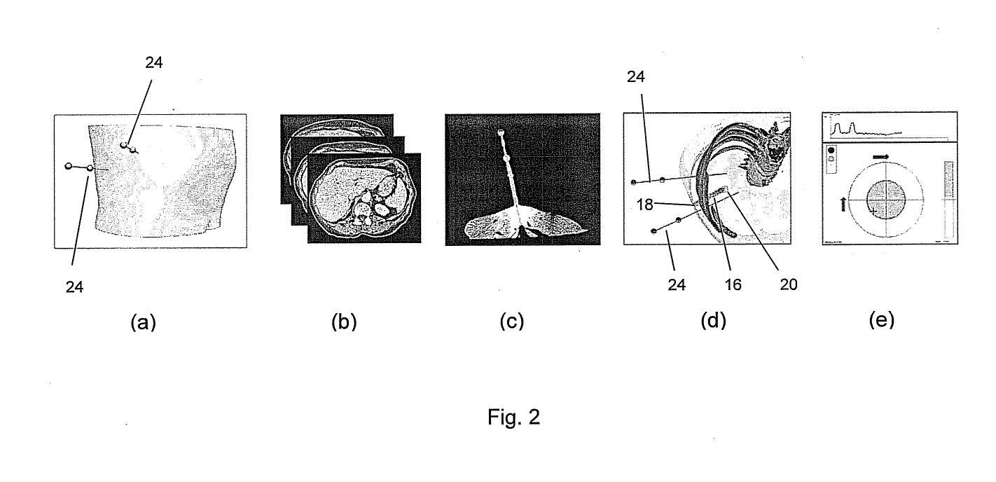

In (a) to (e) of FIG. 2, the workflow of a minimally invasive intervention is schematically summarized in which the method and system of the invention can be employed. By a way of example only, the intervention is considered to be an ablation of a tumour in a human body's liver. The ablation is done with a needle-like elongate instrument having a tip portion that is configured for radiofrequency ablation.

In a first step, schematically shown in (a) of FIG. 2, fiducial needles 24 are inserted into the patient's body part, such that their tips will lie within the liver and in the vicinity of the tumour 10 to be ablated. The fiducial needles 24 have a needle-shaped body with a rotationally symmetric elongate portion serving as a marking portion for tracking. Suitable embodiments of such fiducial needles 24 are described in EP 1 632 194 A1. Custom-designed silicon patches may be used to affix the fiducial needles 24 to the skin of the patient and to prevent them from slipping out. Alternatively, the fiducial needles 24 are fixed in the liver. As has been demonstrated in the article “Soft tissue navigation using needle-shaped markers: Evaluation of navigation aid tracking accuracy and CT registration”, in Proceedings of SPIE Medical Imaging 2007: Visualization, Image-Guided Procedures, and Display, K. R. Cleary and M. I. Miga, eds., 650926 (12 pages) February 2007, L. Maier-Hein, D. Maleike, J. Neuhaus, A. Franz, I. Wolf, and H.-P. Meinzer, such fiducial needles can be constructed precisely to obtain a sub-millimeter tracking accuracy.

In a second step, represented by panel (b) of FIG. 2, the CT image of the patient's body part, i.e. the abdomen containing the liver is taken. In the CT image, the fiducial needles 24 are visible, as is shown in FIG. 2(c). Note that in the general framework of the invention, different types of medical imaging could be used, such as nuclear magnetic resonance (NMR) imaging and ultrasound imaging.

For assisting the physician in targeting the tumour 10, the elongate surgical instrument, i.e. the ablation needle 22 is tracked using a standard tracking system. Suitable tracking systems may be optical and/or electromagnetic systems for continuously locating the position of the ablation needle 22 during the intervention. Optical tracking systems are highly accurate but require a constant line of sight between the tracking system and the tracked sensors. Electro-magnetic systems, on the other hand, are less robust and accurate but allow for integration of the sensors into the tip of the instrument.

To visualize the ablation needle 22 in relation to anatomical structures extracted from the CT image acquired in the second step (b) of FIG. 2, it is necessary to register the tracking coordinate system with the image coordinate system. However, since in the present example, the tumour 10 is located in the soft tissue of the liver 12 which is close to the patient's diaphragm, the tumour will move during the patient's breathing cycle. To perform the registration, in one embodiment one seeks to locate the fiducials 24 in both, the tracking and the CT image coordinate systems in the same motion state of the abdomen, i.e. during matching states within the breathing cycle. Since the needles are inserted into the moving tissue, the motion of the tissue will be reflected in a motion of the fiducial needles 24.

In one embodiment, the fiducial needles are tracked over time to identify the state within the breathing cycle in which the CT image was taken in. For this purpose, two landmarks l̅ⱼ₁⁰, l̅ⱼ₂⁰ from the axis of each registered fiducial needle j are extracted. These landmarks can be the tip of the fiducial needle 24 itself and a second point on the axis of the needle 24 with a certain distance to the tip. Then, the fiducial needles 24 can be tracked over at least one breathing cycle such as to obtain a sequence of tracked needle positions k over time. If two needles 24 are used, for every sample k four landmark vectors acquired: L^{k}={ l̅₁₁^{k}, l̅₁₂^{k}, l̅₂₁^{k}, l̅₂₂^{k}}. For each sample k a rigid transformation Φₖ mapping the current landmarks L^{k} onto the original land-marks L⁰ in the medical image is computed. Then, for each of the samples k a fiducial registration error (FRE) is computed, indicating how well the positions of the fiducial needles in the tracking coordinate system correspond with their positions in the CT image:

$$F R {E}^{k} = \frac{1}{4} {\sum }_{j = 1}^{2} {\sum }_{m = 1}^{2} \vert {{l}^{->}}_{jm}^{0} - {Φ}_{k} ( {{l}^{->}}_{jm}^{k} ) \vert$$

While the FRE in this example is defined as a mean value, it could also be defined as a root-mean-square-error or the like.

The sample k for which the FRE becomes the least corresponds to the state within the breathing cycle which the CT was taken in, and the corresponding coordinate transformation Φₖ for the sample k is chosen as the transformation \hat{Φ} for initial registration.

Note that the purpose of the registration is to calculate the position of the tumour 10 in the tracking coordinate system. Thus, the precision of the computed location of the tumour as compared to its actual position in the tracking coordinate system will depend on the validity of the transformation \hat{Φ} used during the registration. As is clear from the above explanation, the transformation \hat{Φ} will be very reliable if the motion state of the body part is very similar to or identical with the motion state in which the CT image was taken. That is, for small FREs, it can be expected that the computed position of the tumour in the tracking coordinate system is very precise. Accordingly, if the FRE is repeatedly displayed to the user, the user can use it as a confidence value as to how reliable registration actually is.

If, however, the motion state of the body part is different from that of the CT image, for ex-ample during different periods of the breathing cycle, in one embodiment this motion can be compensated mathematically. One way to achieve this is to constantly track the fiducials 24 during the intervention in regular intervals of for example a few tens or a hundred microseconds. In each of these tracking instances, a set of landmarks L_{track}^{cur} be extracted from the tracked fiducial needle positions and transformed to the image coordinates using the trans-formation \hat{Φ} which had been determined as described above. Next, a time dependent, current transformation \hat{Φ}_{cur} can be computed, which maps the original needle positions L⁰ onto the transformed current position L_{ing}^{cur}. Finally, Φ_{cur} can be used to transform the target point \vec{t}₀ to originally located in the planning CT onto the \vec{t}_{cur}:

\vec{t}_{cur}Φ_{cur}(\vec{t}₀)

Different types of real-time compatible transformations can be used for motion compensation, such as thin-plate splines or affine transformations, as are for example described in “Respiratory motion compensation for CT-guided interventions in the liver”, Comp Aid Surg 13(3), pp. 125-38, 2008, L. Maier-Hein, S. A. Müller, F. Pianka, S. Wörz, B. P. Müller-Stich, A. Seitel, K. Rohr, H.-P. Meinzer, B. Schmied, and I. Wolf.

With reference to panel (d) of FIG. 2, next the trajectory 16 for inserting the ablation needle into the body part is planned. This trajectory planning can be performed using the CT image obtained in step (b). Using the CT image, a suitable trajectory 16 can be chosen which connects an entry point 18 with a target point 20, such as the centre of mass of the tumour 10 and which avoids bony structures and risk structures.

After the trajectory 16 has been planned, the physician has to insert the ablation needle 22 along the predetermined trajectory 16 to reach the tumour 10. To achieve this, a navigation monitor is used on which images are displayed that assist the physician to target the tumour 10 while inserting the ablation needle 22 along the predetermined trajectory 16. The navigation monitor can be an ordinary computer display on which the images are displayed. The images can be generated by a computer system, which may for example be an ordinary personal computer, a notebook or a workstation. The computer system may be configured to receive inputs from an ordinary tracking system and is capable to store medical images as acquired in step (b) of FIG. 2. The computer system may comprise a software which when executed on the computer system carries out a method for assisting the targeting of the tumour 10. When such software is installed in the computer system comprised of ordinary or specifically adapted hardware components, a system for computed assisted targeting is materialized.

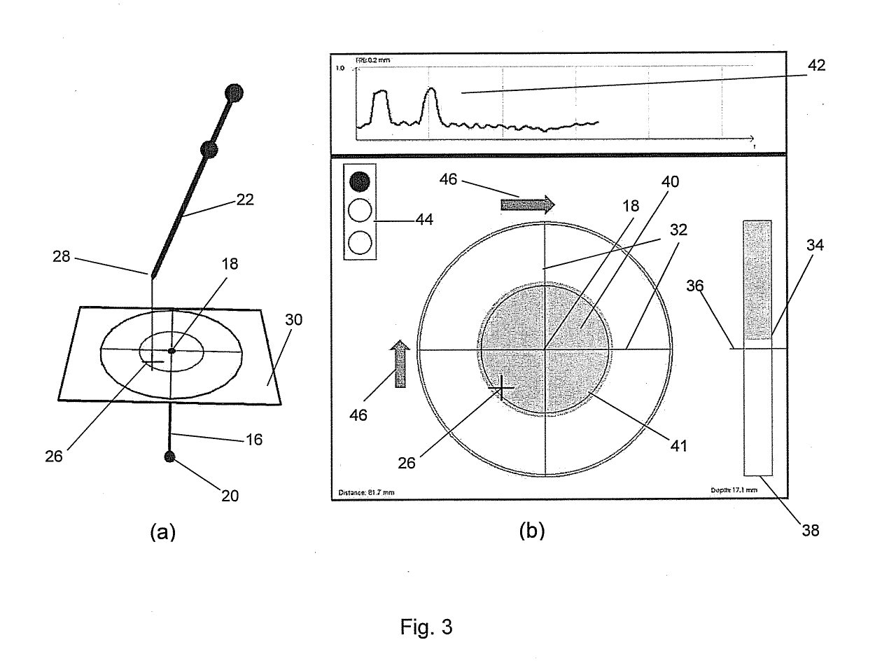

FIG. 3b shows a screenshot of an image generated and displayed by a navigation monitor during an entry point finding assisting step. In this image, a projection 26 of the tip 28 of the ablation needle 22 onto a plane 30 is displayed, which plane includes the predetermined entry point 18 and which is normal to the vector connecting the predetermined entry point 18 and the target point 20 (see FIG. 3a), where the projection is a projection in a direction parallel to this vector or, in other words, parallel to the predetermined trajectory 16. Also displayed in the screenshot of FIG. 3b is the predetermined entry point 18 at the intersection of two lines 32 forming a cross recticle.

Further in the screenshot of FIG. 3b, a depth indicator 34 is displayed. The depth indicator 34 is a bar diagram representing the distance between the tip 28 of the ablation needle 22 and the target point 20 which indicates at which position along the predetermined trajectory the tip 28 of the ablation needle 22 currently is. If the bar of the depth indicator 34 has reached a centre line 36, this indicates that the tip 28 has reached the entry point on the skin of the patient and if the bar has reached the bottom line 38, this indicates that the tip 28 has reached the target point. Also, the depth or distance from the target point 20 can be indicated by circle of variable size 40 surrounding the predetermined entry point. The further the tip 28 is away from the predetermined entry point 18, the larger is the circle 40. If the needle 22 is lowered onto the patient's skin, the circle 40 shrinks just like a light spot of a torchlight approaching a wall. If the distance corresponding to the predetermined entry point 18 is reached, the circle 40 coincides with a stationary circle 41.

In a top portion of FIG. 3b, the fiducial registration error (FRE) is displayed as a function of time. As has been explained above, the FRE directly reflects the breathing cycle of the patient. For example, if the CT image was taken in the fully respirated state, a small FRE reflects a currently respirated state of the patient, where the FRE increases each time the patient inhales. Thus, the FRE as displayed in the diagram 42 of FIG. 3b can be interpreted as a breathing curve.

Further in FIG. 3b, a “signal light” 44 and guiding arrows 46 are displayed, the function of which will be explained below.

The image generated in FIG. 3b is meant to assist the physician in finding the predetermined entry point 18 with the tip 28 of the ablation needle 22. When the physician lowers the tip 28 of the ablation needle 22 onto the skin of the patient, he only has to make sure that the cross-mark 26 representing the projection of the tip 28 onto the plane 30 coincides with the predetermined entry point 18, which is also displayed in FIG. 3b. Thus, the physician only has to move to the tip 28 of the needle parallel to the skin of the patient until the cross-mark 26 and the predetermined entry point 18, i.e. the intersection of the two lines 32 coincide. The two-dimensional information displayed in FIG. 3b is the crucial information for finding the entry point, while the third dimension can be accessed by the physician easily by noticing that the tip 28 of the needle 22 has touched the patient's skin. Also, this third dimension is reflected by the depth indicators 34 and 40. This abstract way of separately displaying the critical two dimensions has been found to greatly assist the physician in finding the predetermined entry point 18.

Guiding arrows 46 indicate in which direction and how far the tip 28 of the instrument has to be moved such as to approach and meet the predetermined entry point 18. There are many alternative ways of displaying information indicating to the user how the tip 28 of the instrument has to be moved such as to approach the predetermined entry point 18, and the present embodiment of FIG. 3b is just an illustrative example. For example, in one embodiment, it would be sufficient to only display the guiding arrows 46 or similar indicators.

Once the predetermined entry point 18 has been found with the predetermined precision, this is indicated by the signal 44, and the entry point finding step is completed.

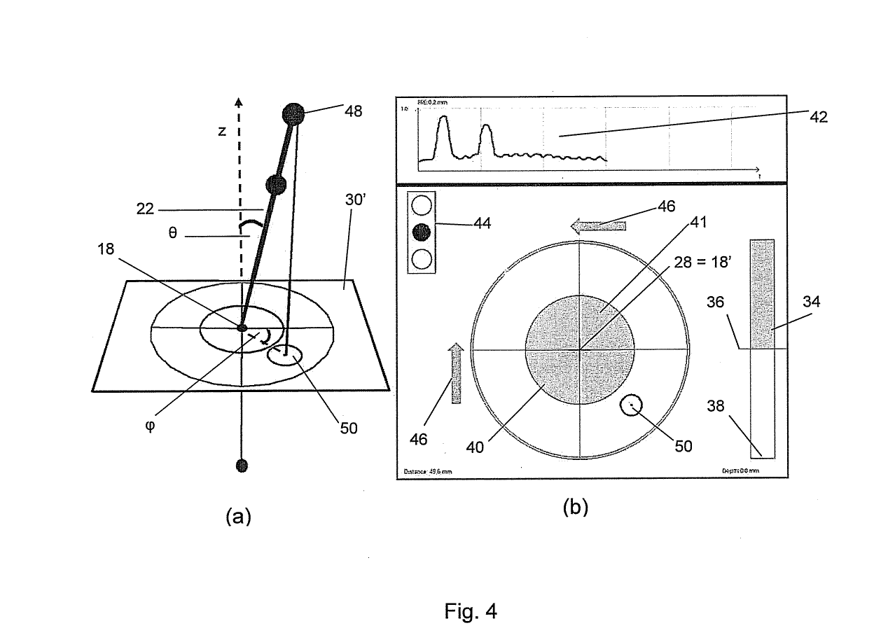

In a next step, the needle 22 shall be aligned with the predetermined trajectory 16. This is assisted by an instrument directing assisting step in which an image as shown in FIG. 4b may be generated and displayed. The image of FIG. 4b is very similar to the one of FIG. 3b, except that this time a projection 50 of an end portion 48 of the ablation needle 22 on a plane 30′ is displayed. Herein, the projection is a projection along a vector connecting the tip portion 28 of the needle 22 and the target point 20, and the plane 30′ is a plane perpendicular to this vector. Since by the time this step is performed, the tip 28 of the needle 22 is meant to be placed at the predetermined entry point, this vector should coincide with the predetermined trajectory 16 and the plane 30′ should be identical with plane 30 shown in FIG. 3a. However, if there should be a small deviation between the actual position of the tip 28 and the predetermined entry point 18, the projection vector and projection plane 30′ used in FIG. 4a allow to correct this error by adjusting the orientation of the needle accordingly.

With reference to FIG. 4a, note that the location of the projection 50 in the plane 30 is actually a representation of the zenith angle θ and the azimuth angle φ of the longitudinal axis of the needle 22 with regard to a z-axis defined by the vector connecting the needle tip 28 and the target point 20. The proper alignment is achieved if the zenith angle θ becomes zero, i.e. if the projection 50 coincides with the position of the needle tip 28, which is represented by the central cross in FIG. 4b and which again is intended to coincide with the predetermined entry point 18. In order to account for a possible small deviation between the actual needle tip 28 and the predetermined entry point 18, the (true) entry point is denoted by 18′ in FIG. 4b.

Again, the image of FIG. 4b only displays the two-dimensional information that is necessary for the user to assess to which extent the longitudinal axis of the instrument 22 is aligned with the vector connecting the target point 20 and the tip portion 28 of the instrument. Note that the distance between the projection 50 and the entry point 18′ is proportion to the sinus of the zenith angle θ, and that perfect alignment is achieved if the needle is tilted such that the projection 50 coincides with the entry point 18′, in which case the zenith angle θ is zero.

While the projection 50 of the end portion 48 is a very intuitive way of representing the zenith and azimuth angle, it goes without saying that there are many different ways to represent these angles which could be used instead. In this disclosure, any two-dimensional image that is related to the zenith and azimuth angle in a one-to-one relationship is regard as a representation” of these angles, and in principle any such representation could be used instead.

Once the projection 50 has been aligned with the entry point 18′, this indicated by the signal light 44, and the needle 22 can be inserted into the patient's body.

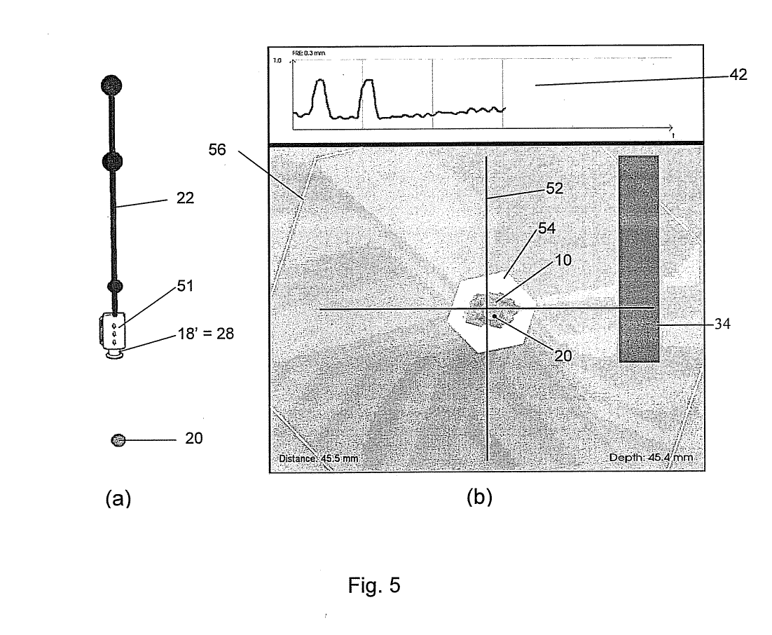

The insertion into the patient's body is assisted by an instrument guiding assisting step in which an image as shown in FIG. 5b is generated and displayed. The image of FIG. 5b is a view of virtual camera 51 placed at the tip of and directed along the longitudinal axis of the ablation needle 22. A schematic view illustrating the concept of the virtual camera 51 is depicted in FIG. 5a. The virtual camera image can be readily computed from a medical image, such as a CT image, registered with the tracking coordinate system.

As the ablation needle 22 is inserted into the body part, the image generated and displayed in FIG. 5a will be a motion picture of a “flight” along the predetermined trajectory 16 towards the tumour 10. A recticle 52 is shown which when coinciding with the target point 20 indicates that the needle 22 is pointing directly to it. While not easily recognizable in the black and white image of FIG. 5b, in one embodiment a virtual tube- or tunnel structure surrounding the predetermined trajectory is displayed, in which the needle has be to kept upon insertion. It has been confirmed in tests that this virtual camera view is a very intuitive way of guiding the instruments which allowed even inexperienced users personnel to guide the needle 22 towards the tumour 10.

Again, a depth indicator 34 is provided from which the user can discern how far needle has to be inserted. Also, upon approaching the tumor with the tip, in the virtual camera view the tumour will appear larger and larger, such that approaching of the tumour is readily recognizable. Or course, the depth indication is crucial for stopping the insertion of the needle at the correct position, such as to not inadvertently penetrate through the tumour 10. To further facilitate finding the correct insertion depth, a polygon-shaped structure 54 surrounding the tumour 10 is shown, which represents the exit plane of the “tunnel” mentioned above. Also, a second polygon 56 is displayed which corresponds to a radial projection of the tip 28 of the instrument onto the wall of the virtual “tunnel”. As the tip 18 of the needle 22 approaches the target point 20, the outer polygon 56 and the inner polygon approach each other, and the outer polygon 56 touches the circumference of the inner polygon 54 just when the end of the “tunnel”, i.e. the predetermined insertion depth is reached. This has been found to greatly assist the physician in delicately controlling the insertion depth up to the target point.

While not shown in FIG. 5b, in one embodiment of the guiding assisting step, images of predetermined objects can be displayed. For example, if there should be a risk structure that has to be avoided upon insertion of the needle 22, such as further tumours, large vessels, further organs and the like, these structures can be included in the image of FIG. 5b, and by visual inspection the user can be constantly sure to keep away from these structures. This greatly reduces the risk of inadvertently encountering risk structures and make the intervention much less dangerous for the patient than the prior art intervention.

The images shown in the example of FIGS. 3b to 5b are images generated by an ordinary computer system on which a computer program according to an embodiment of the invention is installed. In the computer system, medical images in a common format as provided for example by CT apparatuses can be stored, and the computer system is further adapted to receive tracking signals from ordinary tracking equipment. Under the control of the computer program, in one embodiment, images as described above are generated and displayed on an ordinary computer monitor or the like.

If the computer program is executed on a computer system, a method including an entry point finding assisting step as explained in reference to FIG. 3b, an instrument directing assisting step as explained with reference to FIG. 4b and an instrument guiding assisting step as explained with reference 5b can be carried out.

In another aspect, a computer program when executed on a computer system may materialize a system for computed assisted targeting comprising assisted entry point finding means, assisted instrument directing means and assisted instrument guiding means.

In the exemplary embodiment described above, the method steps and assisting means are split up in three separate items each specifically adapted to the corresponding actions to be taken by the physician upon inserting the elongate instrument, namely finding the entry point, directing the instrument such as to point toward the target point and guiding the instrument upon insertion to stay as closely to the predetermined trajectory as possible. However, the steps could also be intermixed in some embodiments. Also, the entry point finding step could be much simpler than the one shown in the specific embodiment. This is particularly true since a deviation between the predetermined entry point and the actual entry point can be fully compensated by the instrument directing assisting step and the instrument guiding assisting step, as has been explained above. Simply put, a deviation from the predetermined trajectory at its beginning (the entry point) is tolerable, as long as it is guaranteed that the end of the trajectory will be exactly at the target point. The second and third steps of the method of the preferred embodiment do guarantee this.

In an alternative embodiment, the entry point finding step could be replaced by an entry point determining step, in which the entry point is only determined during the intervention. For ex-ample, the physician could point with the tip of the instrument on different positions of the skin of the patient such as to proposed trial entry points, and the system could calculate the corresponding trajectory and indicate whether the trajectory would be suitable according to predetermined criteria. One of such predetermined criteria could be that the trial trajectory is sufficiently far away from risk structures or obstructing structures. Once one of the trial entry points has been selected, it plays the role of a “predetermined entry point” as mentioned in the foregoing example, which is therefore applicable to such an embodiment as well.

Also, the entry point finding assisting step could be modified to be a combined finding and determining step. For example, the physician could scan the surface of the patient's skin with the tip of the instrument, and an image could be continuously generated and displayed indicating whether a current position of the instrument during the scanning would give a suitable entry point or not, for example by displaying a predetermined color (such as red for non-suitable entry point and green for suitable entry point). Note that in all of the variants, the instrument directing assisting step and the instrument guiding assisting step remain unaffected and are thus compatible with all these variants.

As further information, in some embodiments a value indicating how well the current positions of the navigation aids 24 correspond with their positions in the medical image is determined and displayed, such as the FRE displayed in panel 42 of FIGS. 3b, 4b and 5b. As explained above, this value can for example represent a breathing curve and allow the physician to perform the insertion in the interval of the breathing cycle that is suited the best.

Also, even when no real-time compensation for soft tissue motion based on the deformation models or the like is provided for, this value may indicate periods of the breathing cycle during which the rigid registration is expected to be very precise, and this allows a physician to perform the insertion process during this period. With reference to the example of the ablation of a tumour 10 in a liver, if the CT image had been taken in an expirated state of the patient, the physician may monitor the FRE value of panel 42 to recognize the onset of the respiration state, perform the entry point finding, needle orientation and the insertion of the needle with the assisted guiding within as may consecutive respiration states as needed.

Also even if a successful means for motion compensation are provided, such that the registration is reliable throughout the breathing cycle, it may still be helpful for the physician to observe the breathing cycle such as to perform the insertion during a period where the tumour is not moving.

The method and system of the invention has been tested in experiments on swines both by medical experts and experience with CT guided interventions and by fourth year medical students which had no such experience. In the experiments, it has been found that the lesion has practically always been hit with the very little error. As a remarkable result, the non-experts performed even better than the experts. A possible explanation for this phenomenon is the fact that the experts are accustomed to inserting the needle very quickly, while the non-experts have to rely to a greater extent on the system described herein, and could therefore more fully exhaust its benefits. This demonstrates that the method and system according to the embodiments of the invention indeed greatly facilitate the targeting of a target, which in turn lowers the risks involved for the patient with this type of invention and also the possible strain involved with repeating the intervention several times if necessary, until the tumour is finally hit, as is of the case in current practice.

Although preferred exemplary embodiment is shown and specified in detail in the drawings and the preceding specification, these should be viewed as purely exemplary and not as limiting the invention. It is noted in this regard that only the preferred exemplary embodiment is shown and specified, and all the variations and modifications are to be protected that presently or in the future lie within the scope of the appended claims.

What is claimed is:

1. A method for assisting the targeting of a target with an elongate instrument, wherein the instrument is to be inserted into a living object's body part along a predetermined trajectory extending between an entry point of said instrument into said body part and a target point associated with said target, said method comprising:
an instrument directing assisting step for generating and displaying an image allowing a user to assess to which extent the longitudinal axis of the instrument is aligned with a vector connecting the tip portion of said instrument and the target point; and
an instrument guiding assisting step of generating and displaying an image allowing a user to assess to which extent the instrument motion during insertion thereof coincides with said predetermined trajectory.

2. The method of claim 1, further comprising an entry point finding assisting step of generating and displaying an image allowing a user to assess how the tip of the instrument has to be moved in order to approach the predetermined entry point.

3. The method of claim 1, further comprising a step of displaying a graphical representation of a parameter representing or related to the distance between said tip portion of the instrument and said target point.

4. The method of claim 2, wherein said image generated and displayed in said entry point assisting step represents a relative position between projections of said predetermined entry point and a tip portion of the instrument along a direction substantially parallel to a vector connecting said entry point and said target point onto a plane.

5. The method of claim 4, wherein said plane on which said predetermined entry point and said tip portion are projected is a plane normal to said vector connecting said predetermined entry point and said target.

6. The method of claim 1, wherein in said instrument directing assisting step the image generated and displayed is an image representative of a zenith angle and an azimuth angle of the longitudinal axis of the instrument with respect to a vector connecting the tip portion of the instrument and the target point.

7. The method of claim 1, wherein in said instrument directing assisting step the two dimensional image displays a projection of a portion of said instrument remote from said tip portion and lying on the instrument's longitudinal axis onto a plane, said plane including said tip portion of the instrument and being perpendicular to a vector connecting said tip portion of said instrument and said target point, said projection being directed along the direction defined by the vector.

8. The method of claim 1, wherein the image generated and displayed in said instrument guiding assisting step corresponds to a view of a virtual camera placed at the tip of and directed along the longitudinal axis of the instrument.

9. The method of claim 8, wherein in said instrument guiding assisting step a tube or tunnel-like structure coaxially surrounding the predetermined trajectory is displayed.

10. The method of claim 8, wherein in said instrument guiding assisting step, medical images of predetermined objects are displayed.

11. The method of claim 10, wherein said predetermined objects comprise one or more of the following: blood vessels, tumors, boney structures, organs.

12. The method of claim 1, further comprising a step of tracking said instrument by optical or electromagnetic tracking means such as to locate the position and orientation of the instrument in a tracking coordinate system.

13. The method of claim 12, further comprising a step of registering said tracking coordinate system with a coordinate system of a medical image of said body part.

14. The method of claim 13, wherein said registering step comprises tracking of navigation aids, such as fiducials, which are provided on or are inserted to the body part and which are recognizable in said medical image.

15. The method of claim 14, wherein said registering step comprises:
tracking the navigation aids during a time interval during which the navigation aids may move along with the body part due to soft tissue motion; and
determining a motion state of the body part in which the positions of the navigation aids coincide best with their positions in the medical image, wherein the registration is performed based on the tracked position of the navigation aids in said determined motion state.

16. The method of claim 15, wherein the motion state corresponds to a certain part of a breathing cycle.

17. The method of claim 1, further comprising a step of repeatedly determining and displaying a value indicating how well the current positions of the navigation aids correspond with their positions in the medical image.

18. The method of claim 1, further comprising a motion compensation step, in which the current position of the target point is calculated based on information about the motion state of the body part.

19. The method of claim 18, wherein said motion compensation step is based on a real-time tracking of the positions of navigation aids and a deformation model for predicting a current deformation of the body part from a current position of said navigation aids.

20. The method of claim 1, further comprising a step of determining an entry point based on a current position of the tip of the instrument.

21. The method of claim 20, further comprising calculating a trajectory connecting said determined entry point and the target point and generating and displaying information indicating whether the trajectory is suitable or not.

22. A system for computed assisted targeting of a target comprising a target point with an elongate instrument, comprising:
assisted instrument directing means for generating and displaying an image allowing a user to assess to which extent the longitudinal axis of the instrument is aligned with a vector connecting a tip portion of said instrument and the target point; and
assisted instrument guiding means for generating and displaying an image allowing a user to assess to which extent the instrument motion during insertion thereof coincides with a predetermined trajectory connecting an entry point of said instrument into a living object's body part and said target point.

23. The system of claim 22, further comprising assisted entry point finding means for generating and displaying an image allowing a user to assess how the tip of said instrument has to be moved in order to approach the predetermined entry point.

24. The system of claim 22, further comprising means for displaying a graphical representation of a parameter representing or related to the distance between said tip portion of the instrument and said target point.

25. The system of claim 24, wherein the image generated and displayed by said assisted entry point finding means represents a relative position between projections of said predetermined entry point and of said tip portion of the instrument along a direction substantially parallel to a vector connecting said predetermined entry point and said target point onto a plane.

26. The system of claim 25, wherein said plane on which said predetermined entry point and said tip portion are projected is a plane normal to said vector connecting said predetermined entry point and said target point.

27. The system of claim 22, wherein the image generated and displayed by the instrument directing means is an image representative of a zenith angle and azimuth angle of the longitudinal axis of the instrument with respect to a vector connecting the tip portion of the instrument and the target point.

28. The system of claim 22, wherein the image generated and displayed by said assisted instrument directing means is a two-dimensional image displaying a projection of a portion of said instrument remote from said tip portion and lying on the instrument's longitudinal axis onto a plane perpendicular to a vector connecting said tip portion of said instrument and said target point.

29. The system of claim 22, wherein said image generated and displayed by said assisted instrument guiding means corresponds to a view of a virtual camera placed at the tip of and directed along the longitudinal axis of the instrument.

30. The system of claim 29, wherein said image generated by said assisted instrument guiding means further comprises a tube- or tunnel-like structure coaxially surrounding the predetermined trajectory.

31. The system of claim 29, wherein said assisted instrument guiding means are further adapted to display medical images of predetermined objects, in particular, but not limited to, blood vessels, tumors, boney structures, organs.

32. The system of claim 22, further comprising means for tracking said instrument based on signals received from optical or electromagnetic tracking means such as to continuously locate the position and orientation of said instrument in a tracking coordinate system.

33. The system of claim 32, further adapted to register said tracking coordinate system with a coordinate system of a medical image of said body part.

34. The system of claim 33, further comprising navigation aids, such as fiducials, to be provided on or inserted to the body part.

35. The system of claim 34, wherein said navigation aids comprise a needle-shaped body having an elongate portion serving as a marker.

36. The system of claim 34, further configured to track said navigation aids during a time interval during which the navigation aids are allowed to move along with the body part due to soft tissue motion, and to determine a motion state of the body part in which the positions of the navigation aids coincide best with their positions in the medical image.

37. The system of claim 34, further configured to repeatedly determine and display a value indicating how well the current positions of the navigation aids correspond with their positions in a given medical image.

38. The system of claim 22, further comprising means for compensating the motion of the soft tissue, said means being configured to calculate a current position of said target point based on information of the motion state of the body part.

39. The system of claim 38, wherein said information of the motion state of the body part is represented by the positions of navigation aids attached to or inserted to the body part, and the calculation is based on a deformation model of the body part.

40. The system of claim 22 further comprising means for determining an entry point based on a current position of the tip of the instrument.

41. The system of claim 40, further comprising means for calculating a trajectory connecting said determined entry point and the target point and generating and displaying information indicating whether the trajectory is suitable or not.

42. A machine readable medium having stored thereon a plurality of executable instructions, the plurality of instructions comprising instructions to:
generate and display an image for assisting a user in finding a predetermined entry point of an elongate instrument into a living object's body part, or determining an entry point;
generate and display an image allowing a user to assess to which extent the longitudinal axis of the elongate instrument is aligned with a vector connecting a tip portion of the instrument with a target point; and
generate and display an image allowing a user to assess to which extent an instrument motion during insertion thereof coincides with a predetermined trajectory connecting said entry point and said target point.
